(19) United States
(12) Patent Application Publication
(10) Pub. No.: US 2005/0208597 A1
(43) Pub. Date: Sep. 22, 2005

(54) Microarray analysis of post-translational modifications

(75) Inventors: Steven M.C. Chan, Mountain View, CA (US); Paul J. Utz, Portola Valley, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: The Board of Trustees of the Leland Stanford Junior University

(21) Appl. No.: 11/040,736

(22) Filed: Jan. 21, 2005

Related U.S. Application Data
(60) Provisional application No. 60/539,444, filed on Jan. 26, 2004.

(51) Int. Cl. G01N 33/53; G01N 33/567

(52) U.S. Cl. 435/7.2

(57) ABSTRACT

Methods and kits are provided for a multiplexed reverse phase protein (RPP) microarray platform, which is utilized for simultaneous monitoring of cellular components following exposure to an agent or agents of interest. The microarray comprises cell lysates or fractions thereof. The array is probed with a specific binding agent or agents. Binding agents of interest include agents specific for inducible proteins, constitutive proteins, apoptosis-specific modifications, etc., and particularly for post-translationally modified proteins, e.g. phosphoproteins, glycosylated proteins, and the like. The methods are of interest for determining patterns of modifications, and the like that define disease states or classify subsets of disease (including staging and subsets of cancers, autoimmune diseases, and the like); that follow response to therapy; that determine response patterns after exposure to a specific agent, and the like. This information is useful in the development of therapies, as a disease prognostic, determining patient specific therapies, and the like. Kits may comprise microarrays having a cell selection of interest, and may further comprise antibodies of desired specificity, and the like.

### BACKGROUND

A variety of post translational modifications of proteins take place. These modifications include phosphorylation, glycosylation, prenylation, and the like. The modifications, particularly reversible phosphorylation, can be a molecular mechanism by which intracellular signals are transmitted. A substantial number of signaling proteins are kinases or phosphatases that act on serine, threonine, and tyrosine residues. With over 2000 human genes predicted to code for kinases and the potential for each kinase to act on multiple targets, signaling networks are immensely complex. An important step towards unraveling this complexity is the development of new proteomics technologies that can quantitatively monitor the phosphorylation states of signaling proteins in a multiplex fashion. Such technologies would enable the detailed analysis of signaling pathways in a global perspective and the rapid identification of previously unrecognized signaling events.

Microarrays offer an attractive and convenient platform for multiplex protein analysis. However, despite a rapidly growing interest in adapting DNA microarray technologies for proteins, the development of protein microarrays for profiling post-translational modification events such as phosphorylation has been slow. Approaches that rely on immobilized antibodies to capture their analytes of interest from solution are constrained by the lack of commercial antibodies that function in this format. Furthermore the detection of bound proteins can be problematic.

As an alternative technology, reverse phase protein (RPP) microarrays have been developed. RPP microarrays are constructed by depositing small volumes of cell lysates onto a high protein-binding substratum using a robotic microarrayer. Each cell lysate microspot contains the full complement of intracellular proteins and analytes from that sample. The arrays are then probed with antibodies, and the signal intensity of each microspot correlates with the level of the analyte. Since thousands of samples can be spotted in high density onto a single slide, a large number of samples can be monitored simultaneously thereby increasing throughput, and simplifying cross-comparisons between samples.

Publications

Robinson et al., Nat Med 8, 295 (March, 2002); Robinson et al., Nat Biotechnol 21, 1033 (September, 2003); MacBeath, Nat Genet 32 Suppl, 526 (December, 2002); Paweletz et al., Oncogene 20, 1981 (Apr. 12, 2001); Espina et al., Proteomics 3, 2091 (November, 2003); Liotta et al., Cancer Cell 3, 317 (Apr, 2003).

### SUMMARY OF THE INVENTION

Methods and kits are provided for a multiplexed reverse phase protein (RPP) microarray platform, which is utilized for simultaneous monitoring of cellular components. Of particular interest are components affected by post-translational modification, and more particularly signaling pathway components. The microarray comprises cell lysates, where the cells may be cell lines, cells isolated from patients, tissue samples, laser capture microdissection of disease tissues, including disease tissues other than cancer, e.g. infiltrating T or B lymphocytes in organs targeted by inflammation, inflamed organ tissues, e.g. B cells in pancreas; and the like. Whole lysates may be used, or fractions thereof, e.g. nuclear fractions, cytoplasmic fractions, postnuclear supernatant fractions, ER fractions, mitochondria fractions, membrane fractions; and the like. Usually the cells will have been exposed to agents of interest, e.g. pharmaceutical agents, cytokines, antigenic stimulation, cross linking of surface receptors, apoptosis-inducing stimuli; transfected with exogenous genes, and the like. Preferably, the microarray comprises lysates from cells before and after such exposure, and may comprise a time course following the events after exposure.

The array is probed with a specific binding agent or agents. Binding agents of interest include defined antibodies, including antibodies specific for inducible proteins, constitutive proteins, apoptosis-specific modifications, etc., and particularly for post-translationally modified proteins, e.g. phosphoproteins, glycosylated proteins, and the like. The binding agents may also be patient serum and other undefined antibody compositions. Binding agents may also include lectins; aptamers, labeled ligands and other binding partner; and other ligands specific for cellular components.

The methods of the invention are of interest for determining patterns of modifications, and the like that define disease states or classify subsets of disease (including staging and subsets of cancers, autoimmune diseases, and the like); that follow response to therapy; that determine response patterns after exposure to a specific agent, and the like. This information is useful in the development of therapies, as a disease prognostic, determining patient specific therapies, and the like. Kits may comprise microarrays having a cell selection of interest, and may further comprise antibodies of desired specificity, and the like.

### BRIEF DESCRIPTION OF THE DRAWINGS

FIG. 1. Protein microarray performance characteristics and validation. (A) Four purified antigens spotted in triplicate over six 2-fold dilutions starting at 900 pg per spot and probed with their corresponding antibodies. (B) Quantitative comparison between Western immunoblotting and protein microarray for measuring Hsp70 induction in heat-shocked Jurkat T cells. (C) Detection of changes in phosphorylation in signaling proteins in PMA treated vs. untreated Jurkat T cells. Corresponding immunoblots of the same samples are shown on the right. Antibodies specific for p44/42 MAPK, MEK1/2 and Akt are phosphorylation-state dependent. Antibodies for SLP-76 and β-actin are not dependent on phosphorylation.

FIG. 2. Applications of reverse-phase protein microarrays. (A) Kinetics of PLCγ1 (Y783) phosphorylation in Jurkat T cells following stimulation with anti-CD3 (pink triangle), anti-CD28 (green circle), both antibodies (blue squares), or isotype control antibodies (empty squares) over 30 minutes. Left panel: Phosphorylation kinetics unadjusted for total PLCγ1 levels. Middle Panel: Total PLCγ1 levels. Right panel: Ratiometric data on phosphorylation kinetics (phospho-PLCγ1/total PLCγ1). See text for details. (B) Phosphorylation kinetics of four signaling proteins (PLCγ1, Akt, MEK1/2, and p44/42 MAPK) in wildtype Jurkat T cells (black square) and J.gamma1, a mutant Jurkat line deficient in PLCγ1 (empty circle), stimulated with both anti-CD3 and CD28 antibodies. Both cell lines displayed similar levels of surface CD3 and CD28 as measured by flow cytometry (data not shown). (C) Proof-of-concept experiment demonstrating the use of protein microarrays for screening compounds with kinase inhibition activity. Jurkats T cells were stimulated with either anti-CD3 or anti-CD28 antibodies in the presence of one of three kinase inhibitors (see text for details). Lysates were collected after 10 minutes of stimulation, spotted on slides, and probed with four phospho-specific antibodies. Representative spots of p-p44/42 MAPK and p-MEK1/2 are shown for anti-CD3 stimulated cells and p-Akt (Ser473) and p-PDK1 for anti-CD28 treated cells.

FIG. 3. Protein microarray screen with a panel of 62 phospho-specific antibodies. (A) List of phosphoproteins with a significant change in phosphorylation following CD3 stimulation alone or dual stimulation through CD3 and CD28. Corresponding immunoblots for phosphoproteins shown in italics are absent because bands of the correct molecular weight were not detected for those proteins. “-” denotes that the change in phosphorylation for the phosphoprotein was not found to be significant. (B) Corresponding immunoblots for the phosphoproteins on the list. (C) For phospho-specific antibodies that recognize phosphorylated substrate motifs, full-length immunoblots are shown. Left to right lane: unstimulated; anti-CD3; anti-CD3 and CD28.

FIG. 4. Raf-1 dephosphorylation induced by CD3 crosslinking. (A) Kinetics of phosphorylation of Raf-1 (Ser259), MEK1/2 and p44/42 MAPK following CD3 (red triangle), CD28 (green circle), CD3/CD28 (blue square) or isotype control (empty square) stimulation. Raf-1 dephosphorylation coincides with MEK1/2 phosphorylation and activation. (B) Peripheral human T lymphocytes were isolated and stimulated with the indicated antibodies. Equal amounts of lysate were loaded on each lane. Immunoblots for phosphorylated Raf-1 (Ser259) and total Raf-1 are shown.

### DETAILED DESCRIPTION OF THE EMBODIMENTS

Methods and kits are provided for a multiplexed reverse phase protein (RPP) microarray platform, which is utilized for simultaneous monitoring of cellular components, particularly components susceptible to post-translational modification. The microarray comprises cell lysates, or fractions thereof. By probing the microarray with specific binding partners to a component of interest, the cells can be characterized with respect to their response to stimulus in the environment, particularly responses that involve post-translational modifications.

### Post-Translational Modification

Glycosylation. Among the post-translational modifications that can be probed, are protein specific glycoslyation. Membrane associated carbohydrate is exclusively in the form of oliogsaccharides covalently attached to proteins forming glycoproteins, and to a lesser extent covalently attached to lipid forming the glycolipids. Glycoproteins consist of proteins covalently linked to carbohydrate. The predominant sugars found in glycoproteins are glucose, galactose, mannose, fucose, GaINAc, GIcNAc and NANA. The distinction between proteoglycans and glycoproteins resides in the level and types of carbohydrate modification. The carbohydrate modifications found in glycoproteins are rarely complex: carbohydrates are linked to the protein component through either O-glycosidic or N-glycosidic bonds. The N-glycosidic linkage is through the amide group of asparagine. The O-glycosidic linkage is to the hydroxyl of serine, threonine or hydroxylysine. The linkage of carbohydrate to hydroxylysine is generally found only in the collagens. The linkage of carbohydrate to 5-hydroxylysine is either the single sugar galactose or the disaccharide glucosylgalactose. In ser- and thr-type O-linked glycoproteins, the carbohydrate directly attached to the protein is GaINAc. In N-linked glycoproteins, it is GIcNAc.

The predominant carbohydrate attachment in glycoproteins of mammalian cells is via N-glycosidic linkage. N-linked glycoproteins all contain a common core of carbohydrate attached to the polypeptide. This core consists of three mannose residues and two GIcNAc. A variety of other sugars are attached to this core and comprise three major N-linked families: High-mannose type contains all mannose outside the core in varying amounts; hybrid type contains various sugars and amino sugars; complex type is similar to the hybrid type, but in addition, contains sialic acids to varying degrees.

Acylation. Many proteins are modified at their N-termini following synthesis. In most cases the initiator methionine is hydrolyzed and an acetyl group is added to the new N-terminal amino acid. Some proteins have the 14 carbon myristoyl group added to their N-termini. The donor for this modification is myristoyl-CoA. This latter modification allows association of the modified protein with membranes. For example, the catalytic subunit of cyclicAMP-dependent protein kinase (PKA) is myristoylated.

Methylation. Post-translational methylation occurs at lysine residues in some proteins such as calmodulin and cytochrome c. The activated methyl donor is S-adenosylmethionine.

Phosphorylation. Post-translational phosphorylation is one of the most common protein modifications that occurs in animal cells. The vast majority of phosphorylations occur as a mechanism to regulate the biological activity of a protein and as such are transient. In animal cells serine, threonine and tyrosine are the amino acids subject to phosphorylation. The largest group of kinases are those that phosphorylate either serines or threonines and as such are termed serine/threonine kinases. The ratio of phosphorylation of the three different amino acids is approximately 1000/100/1 for serine/threonine/tyrosine. Although the level of tyrosine phosphorylation is minor, the importance of phosphorylation of this amino acid is profound. As an example, the activity of numerous growth factor receptors is controlled by tyrosine phosphorylation.

Sulfation. Sulfate modification of proteins occurs at tyrosine residues such as in fibrinogen and in some secreted proteins, e.g. gastrin. The universal sulfate donor is 3′-phosphoadenosyl-5′-phosphosulphate (PAPS).

Prenylation. Prenylation refers to the addition of the 15 carbon farnesyl group or the 20 carbon geranylgeranyl group to acceptor proteins, both of which are isoprenoid compounds derived from the cholesterol biosynthetic pathway. The isoprenoid groups are attached to cysteine residues at the carboxy terminus of proteins in a thioether linkage (C—S—C). A common consensus sequence at the C-terminus of prenylated proteins has been identified and is composed of CAAX, where C is cysteine, A is any aliphatic amino acid (except alanine) and X is the C-terminal amino acid. In order for the prenylation reaction to occur the three C-terminal amino acids (AAX) are first removed and the cysteine activated by methylation in a reaction utilizing S-adenosylmethionine as the methyl donor. Important examples of prenylated proteins include the oncogenic GTP-binding and hydrolyzing protein Ras and the g-subunit of the visual protein transducin, both of which are farnesylated. Numerous GTP-binding and hydrolyzing proteins (termed G-proteins) of signal transduction cascades have g-subunits modified by geranylgeranylation.

Vitamin C-Dependent Modifications. Modifications of proteins that depend upon vitamin C as a cofactor include proline and lysine hydroxylations and carboxy terminal amidation. The hydroxylating enzymes are identified as prolyl hydroxylase and lysyl hydroxylase. The donor of the amide for C-terminal amidation is glycine. The most important hydroxylated proteins are the collagens. Several peptide hormones such as oxytocin and vasopressin have C-terminal amidation.

Vitamin K-Dependent Modifications. Vitamin K is a cofactor in the carboxylation of glutamic acid residues. The result of this type of reaction is the formation of a γ-carboxyglutamate (gamma-carboxyglutamate), referred to as a gla residue. The formation of gla residues within several proteins of the blood clotting cascade is critical for their normal function. The presence of gla residues allows the protein to chelate calcium ions and thereby render an altered conformation and biological activity to the protein. The coumarin-based anticoagulants, warfarin and dicumarol function by inhibiting the carboxylation reaction. back to the top.

Selenoproteins. Selenium is a trace element and is found as a component of several prokaryotic and eukaryotic enzymes that are involved in redox reactions. The selenium in these selenoproteins is incorporated as a unique amino acid, selenocysteine, during translation. A particularly important eukaryotic selenoenzyme is glutathione peroxidase. This enzyme is required during the oxidation of glutathione by hydrogen peroxide (H₂O₂) and organic hydroperoxides. Incorporation of selenocysteine by the translational machinery occurs via an interesting and unique mechanism. The tRNA for selenocysteine is charged with serine and then enzymatically selenylated to produce the selenocysteinyl-tRNA. The anticodon of selenocysteinyl-tRNA interacts with a stop codon in the mRNA (UGA) instead of a serine codon. The selenocysteinyl-tRNA has a unique structure that is not recognized by the termination machinery and is brought into the ribosome by a dedicated specific elongation factor. An element in the 3′ non-translated region (UTR) of selenoprotein mRNAs determines whether UGA is read as a stop codon or as a selenocysteine codon.

### Arrays

Substrate. Any surface to which the cell lysates of the subject invention are attached, where the cell lysates or fractions thereof are attached in a pre-determined spatial array of arbitrary shape. The array may comprise a plurality of different cell lysates or fractions there, which are patterned in a pre-determined manner, including duplicates of single types.

A variety of solid supports or substrates are suitable for the purposes of the invention, including both flexible and rigid substrates. By flexible is meant that the support is capable of being bent, folded or similarly manipulated without breakage. Examples of flexible solid supports include acrylamide, nylon, nitrocellulose, polypropylene, polyester films, such as polyethylene terephthalate, etc. Also included are gels, e.g. collagen gels, matrigels, and ECM gels. Rigid supports do not readily bend, and include glass, fused silica, quartz, plastics, e.g. polytetrafluoroethylene, polypropylene, polystyrene, polycarbonate, and blends thereof, and the like; metals, e.g. gold, platinum, silver, and the like; etc. In addition, a rigid support may also incorporate a multi-electrode-array for electrical recording and stimulation or any other construct of interest onto which cues could be dispensed.

Derivitized and coated slides are of interest. Such slides are commercially available, or may be produced using conventional methods. For example, SuperAldehyde™ substrates contain primary aldehyde groups attached covalently to a glass surface. Coated-slides include films of nitrocellulose (FastSlides™, Schleicher & Schuell), positively-charged nylon membranes (CastSlides™, Schleicher & Schuell), and a polyacrylamide matrix (HydroGel™, Packard Bioscience), etc.

The substrates can take a variety of configurations, including filters, fibers, membranes, beads, particles, dipsticks, sheets, rods, etc., usually a planar or planar three-dimensional geometry is preferred. The materials from which the substrate can be fabricated should ideally exhibit a low level of non-specific binding during binding events, except for specific cases, in which some non-specific binding is preferred.

In one embodiment of the invention, the substrate comprises a planar surface, and the binding members are spotted on the surface in an array. The binding member spots on the substrate can be any convenient shape, but will often be circular, elliptoid, oval or some other analogously curved shape. The local density of the spots on the solid surface can be at least about 500/cm² and usually at least about 1000/cm² but does not exceed about 10,000/cm², and usually does not exceed about 5000/cm². The spot to spot distance (center to center) is usually from about 100 μm to about 200 μm. The spots can be arranged in any convenient pattern across or over the surface of the support, such as in rows and columns so as to form a grid, in a circular pattern, and the like, where generally the pattern of spots will be present in the form of a grid across the surface of the solid support.

The subject substrates can be prepared using any convenient means. One means of preparing the supports is to synthesize the binding members, and then deposit as a spot on the support surface. The binding members can be prepared using any convenient methodology, such as automated solid phase synthesis protocols, monoclonal antibody culture, isolation from serum, recombinant protein technology and like, where such techniques are known in the art. The prepared binding members can then be spotted on the support using any convenient methodology, including manual techniques, e.g. by micro pipette, ink jet, pins, etc., and automated protocols. Of particular interest is the use of an automated spotting device, such as the Beckman Biomek 2000 (Beckman Instruments). A number of contact and non-contact microarray printers are available and may be used to print the binding members on a substrate. For example, non-contact printers are available from Perkin Elmer (BioChip Arrayer™, Packard). Contact printers are commercially available from TeleChem International (ArrayIt™). Non-contact printers are of particular interest because they are more compatible with soft/flexible surfaces.

The total number of binding member spots on the substrate will vary depending on the number of different binding probes and conditions to be explored, as well as the number of control spots, calibrating spots and the like, as may be desired. Generally, the pattern present on the surface of the support will comprise at least about 10 distinct spots, usually at least about 200 distinct spots, and more usually at least about 500 distinct spots, where the number of spots can be as high as 50,000 or higher, but will usually not exceed about 25,000 distinct spots, and more usually will not exceed about 15,000 distinct spots. Each distinct probe composition may be present in duplicate or more (usually, at least 5 replicas) to provide an internal correlation of results. Also, for some tasks (such as stem cell fate manipulation and other cases, in which a group of cells tend to grow and occupy several spots) it is desirable to replicate blocks, each of several identical spots.

Cells. Cells for use in the assays of the invention can be an organism, a single cell type derived from an organism, or can be a mixture of cell types, as is typical of in vivo situations, but may be the different cells present in a specific environment, e.g. vessel tissue, liver, spleen, heart muscle, brain tissue, etc.

The invention is suitable for use with any cell type, including primary cells, biopsy tissue, normal and transformed cell lines, transduced cells and cultured cells, which can be single cell types or cell lines; or combinations thereof. In assays, cultured cells may maintain the ability to respond to stimuli that elicit a response in their naturally occurring counterparts. Cultured cells may have gone through up to five passages or more, sometimes 10 passages or more. These may be derived from all sources, particularly mammalian, and with respect to species, e.g., human, simian, rodent, etc., although other sources of cells may be of interest in some instances, such as plant, fungus, etc.; tissue origin, e.g. heart, lung, liver, brain, vascular, lymph node, spleen, pancreas, thyroid, esophageal, intestine, stomach, thymus, etc.

In addition, cells that have been genetically altered, e.g. by transfection or transduction with recombinant genes or by antisense technology, to provide a gain or loss of genetic function, may be utilized with the invention. Methods for generating genetically modified cells are known in the art, see for example “Current Protocols in Molecular Biology”, Ausubel et al., eds, John Wiley & Sons, New York, N.Y., 2000. The genetic alteration may be a knock-out, usually where homologous recombination results in a deletion that knocks out expression of a targeted gene; or a knock-in, where a genetic sequence not normally present in the cell is stably introduced.

A variety of methods may be used in the present invention to achieve a knock-out, including site-specific recombination, expression of anti-sense or dominant negative mutations, and the like. Knockouts have a partial or complete loss of function in one or both alleles of the endogenous gene in the case of gene targeting. Preferably expression of the targeted gene product is undetectable or insignificant in the cells being analyzed. This may be achieved by introduction of a disruption of the coding sequence, e.g. insertion of one or more stop codons, insertion of a DNA fragment, etc., deletion of coding sequence, substitution of stop codons for coding sequence, etc. In some cases the introduced sequences are ultimately deleted from the genome, leaving a net change to the native sequence.

Different approaches may be used to achieve the “knock-out”. A chromosomal deletion of all or part of the native gene may be induced, including deletions of the non-coding regions, particularly the promoter region, 3′ regulatory sequences, enhancers, or deletions of gene that activate expression of the targeted genes. A functional knock-out may also be achieved by the introduction of an anti-sense construct that blocks expression of the native genes. “Knock-outs” also include conditional knock-outs, for example where alteration of the target gene occurs upon exposure of the animal to a substance that promotes target gene alteration, introduction of an enzyme that promotes recombination at the target gene site (e.g. Cre in the Cre-lox system), or other method for directing the target gene alteration.

A genetic construct may be introduced into tissues or host cells by any number of routes, including calcium phosphate transfection, viral infection, microinjection, or fusion of vesicles. Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the DNA, then bombarded into cells.

Cell types that can find use in the subject invention include stem and progenitor cells, e.g. embryonic stem cells, hematopoietic stem cells, mesenchymal stem cells, neural crest cells, etc., endothelial cells, muscle cells, myocardial, smooth and skeletal muscle cells, mesenchymal cells, epithelial cells; hematopoietic cells, such as lymphocytes, including T-cells, such as Th1 T cells, Th2 T cells, Th0 T cells, cytotoxic T cells; B cells, pre-B cells, etc.; monocytes; dendritic cells; neutrophils; and macrophages; natural killer cells; mast cells; etc.; adipocytes, cells involved with particular organs, such as thymus, endocrine glands, pancreas, brain, such as neurons, glia, astrocytes, dendrocytes, etc. and genetically modified cells thereof. Hematopoietic cells may be associated with inflammatory processes, autoimmune diseases, etc., endothelial cells, smooth muscle cells, myocardial cells, etc. may be associated with cardiovascular diseases; almost any type of cell may be associated with neoplasias, such as sarcomas, carcinomas and lymphomas; liver diseases with hepatic cells; kidney diseases with kidney cells; etc.

The cells may also be transformed or neoplastic cells of different types, e.g. carcinomas of different cell origins, lymphomas of different cell types, etc. The American Type Culture Collection (Manassas, Va.) has collected and makes available over 4,000 cell lines from over 150 different species, over 950 cancer cell lines including 700 human cancer cell lines. The National Cancer Institute has compiled clinical, biochemical and molecular data from a large panel of human tumor cell lines, these are available from ATCC or the NCI (Phelps et al. (1996) Journal of Cellular Biochemistry Supplement 24:32-91). Included are different cell lines derived spontaneously, or selected for desired growth or response characteristics from an individual cell line; and may include multiple cell lines derived from a similar tumor type but from distinct patients or sites.

Cells may be non-adherent, e.g. blood cells including monocytes, T cells, B-cells; tumor cells, etc., or adherent cells, e.g. epithelial cells, endothelial cells, neural cells, etc. In order to profile adherent cells, they must be dissociated from the substrate that they are adhered to, and from other cells, in a manner that maintains their ability to recognize and bind to probe molecules. Methods of dissociating cells are known in the art, including protease digestion, etc. Preferably the dissociation methods use enzyme-free dissociation media.

Lysates. The cells, which may be cells after exposure to an agent or condition of interest, are lysed prior to spotting on the substrate. Methods of lysis are known in the art, including sonication, non-ionic surfactants, etc. Non-ionic surfactants include the Triton™ family of detergents, e.g. Triton™ X-15; Triton™ X-35; Triton™ X-45; Triton™ X-100; Triton™ X-102; Triton™ X-114; Triton™ X-165, etc. Brij™ detergents are also similar in structure to Triton™ X detergents in that they have varying lengths of polyoxyethylene chains attached to a hydrophobic chain. The Tween™ detergents are nondenaturing, nonionic detergents, which are polyoxyethylene sorbitan esters of fatty acids. Tween™ 80 is derived from oleic acid with a C₁₈ chain while Tween™ 20 is derived from lauric acid with a C₁₂ chain. The zwitterionic detergent, CHAPS, is a sulfobetaine derivative of cholic acid. This zwitterionic detergent is useful for membrane protein solubilization when protein activity is important. The surfactant is contacted with the cells for a period of time sufficient to lyse the cells and remove additional adherent cells from the system.

Methods of cellular fractionation are also known in the art. Subcellular fractionation consists of two major steps, disruption of the cellular organization (lysis) and fractionation of the homogenate to separate the different populations of organelles. Such a homogenate can then be resolved by differential centrifugation into several fractions containing mainly (1) nuclei, heavy mitochondria, cytoskeletal networks, and plasma membrane; (2) light mitochondria, lysosomes, and peroxisomes; (3) Golgi apparatus, endosomes and microsomes, and endoplasmic reticulum (ER); and (4) cytosol. Each population of organelles is characterized by size, density, charge, and other properties on which the separation relies.

Centrifugation is an effective method for organelle isolation. Several other techniques that exploit various physical parameters, e.g. electrical charge for free flow electrophoresis; or biological properties, e.g., ligand affinity for immunoisolation, are also useful for isolating complex organelles and membranes. However, centrifugation is easily set up and ideally combined with analytical proteomics techniques. PNS obtained in the first centrifugation step after the homogenization of cells can be additionally fractionated by different means.

A very simple and rapid fractionation protocol represents high-speed sedimentation/centrifugation (100 000 g), which separates the total membrane fraction from all soluble proteins. This method is very robust and can also be used with small sample volumes in tabletop ultracentrifuges or mini-rotors with a conventional airfuge. This protocol allows fractionation of cells into three major constituents, membranes, cytosol, and nuclei. It is suitable for the overall analysis of quantitative changes of proteins as well as for identification of their posttranslational modifications brought about by growth, differentiation, senescence, environmental changes, genetic manipulation, or other events.

Alternatively, the PNS can be additionally fractionated by density gradient centrifugation. The position of membrane particles in density gradients is determined mainly by the ratio of their lipid to protein content; e.g. mitochondrial inner membranes are protein-rich and thus have a high density, whereas endosomal membranes are lipid-rich and are of low density. Other parameters that determine density include the contents of vesicles. For example, secretory low-density lipoproteins contained within Golgi vesicles render them more buoyant, whereas the protein contents of secretory granules increases their density (eg, pituitary secretory vesicles). The presence of attached components (e.g., ribosomes on rough-ER membranes and clathrin on coated vesicles) also affects the density of membranes. Although differences in composition of subcellular components affect relative densities of fractions, the degree of separation obtained also depends on the nature of the gradient medium used. Sucrose is the most commonly used gradient medium, but there are many other alternatives, eg, Ficoll, Percoll, Nycodenz, or Metrizamide.

Discontinuous gradients as well as step gradients may be used. For better resolution, equilibrium separations with continuous gradients are the method of choice. After centrifugation to equilibrium, membranes distribute throughout the entire gradient according to their specific densities.

### Specific Binding Partners

The term “specific binding member” as used herein refers to a member of a specific binding pair, i.e. two molecules, usually two different molecules, where one of the molecules through chemical or physical means specifically binds to the other molecule. The complementary members of a specific binding pair are sometimes referred to as a ligand and receptor; or receptor and counter-receptor.

Binding pairs of interest include antigen and antibody specific binding pairs, peptide-MHC-antigen complexes and T cell receptor pairs, biotin and avidin or streptavidin; carbohydrates and lectins; complementary nucleotide sequences; peptide ligands and receptor; effector and receptor molecules; hormones and hormone binding protein; enzyme cofactors and enzymes; enzyme inhibitors and enzymes; and the like. The specific binding pairs may include analogs, derivatives and fragments of the original specific binding member. For example, an antibody directed to a protein antigen may also recognize peptide fragments, chemically synthesized peptidomimetics, labeled protein, derivatized protein, etc. so long as an epitope is present.

Immunological specific binding pairs include antigens and antigen specific antibodies; and T cell antigen receptors, and their cognate MHC-peptide conjugates. Suitable antigens may be haptens, proteins, peptides, carbohydrates, etc. Recombinant DNA methods or peptide synthesis may be used to produce chimeric, truncated, or single chain analogs of either member of the binding pair, where chimeric proteins may provide mixture(s) or fragment(s) thereof, or a mixture of an antibody and other specific binding members. Antibodies and T cell receptors may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art.

The binding member may be directly or indirectly labeled with an optically detectable label. Of particular interest as a label are fluorophores. Fluorescence is a physical phenomenon based upon the ability of some molecules to absorb and emit light. With some molecules, the absorption of light at specified wavelengths is followed by the emission of light from the molecule of a longer wavelength and at a lower energy state. Such emissions are called fluorescence and the emission lifetime is said to be the average period of time the molecule remains in an excited energy state before it emits light of the longer wavelength. Substances that release significant amounts of fluorescent light are termed “fluorophores”. This broad class includes fluorescein isothiocyanate (FITC), fluorescein di-galactose (FDG); lissamine, rhodamine, Texas Red, phycoerythrin, allophycocyanin, 6-carboxyfluorescein (6-FAM), 2,7-dimethoxy-4,5-dichloro-6-carboxyfluorescein (6-JOE), 6-carboxy-X-rhodamine (6-ROX), 6-carboxy-2,4,4′,5′,7,7′-hexachlorofluorescein (6-HEX), 5-carboxyfluorescein (5-FAM) or N,N,N,N-tetramethyl-6-carboxyrhodamine (6-TAMRA); dansyl chloride; naphthylamine sulfonic acids such as 1-anilino-8-naphthalene sulfonic acid (“ANS”) and 2-p-toluidinylnaphthalene-6-sulfonic acid (“TNS”) and their derivatives; acridine orange; proflavin; ethidium bromide; quinacrine chloride; and the like.

Highly luminescent semiconductor quantum dots (zinc sulfide-capped cadmium selenide) have been covalently coupled to biomolecules for use in ultrasensitive biological detection (Stupp et al. (1997) Science 277(5330):1242-8; Chan et al. (1998) Science 281(5385):2016-8). Compared with conventional fluorophores, quantum dot nanocrystals have a narrow, tunable, symmetric emission spectrum and are photochemically stable (Bonadeo et al. (1998) Science 282(5393):1473-6). The advantage of quantum dots is the potential for exponentially large numbers of independent readouts from a single source or sample.

### Parameters

The specific binding partner will bind with a cellular parameter of interest. Parameters may include a variety of post-translational modifications, e.g. phosphoserine, phosphotyrosine; acyl groups, etc. In addition to, or in combination with, a parameter can be any cell component or cell product including receptor, protein or conformational or posttranslational modification thereof, lipid, carbohydrate, organic or inorganic molecule, nucleic acid, e.g. mRNA, DNA, etc. or a portion derived from such a cell component or combinations thereof. Parameters may provide a quantitative readout, in some instances a semi-quantitative or qualitative result.

Parameters of interest include detection of cytoplasmic biomolecules, frequently biopolymers, e.g. polypeptides, polysaccharides, polynucleotides, lipids, etc. In one embodiment, parameters include specific epitopes. Epitopes are frequently identified using specific monoclonal antibodies or receptor probes. A parameter may be defined by a specific monoclonal antibody or a ligand or receptor binding determinant.

Microarrays can be scanned to detect binding of the cellular components, e.g. by using a simple light microscopy, scanning laser microscope, by fluorimetry, a modified ELISA plate reader, etc. For example, a scanning laser microscope may perform a separate scan, using the appropriate excitation line, for each of the fluorophores used. The digital images generated from the scan are then combined for subsequent analysis. For any particular array element, the ratio of the fluorescent signal with one label is compared to the fluorescent signal from the other label DNA, and the relative abundance determined.

### Agents

Of particular interest for the methods of the invention are cells before, after and/or during exposure to an agent or agents of interest. Candidate biologically active agents may encompass numerous chemical classes, primarily organic molecules, which may include organometallic molecules, inorganic molecules, genetic sequences, etc. An important aspect of the invention is to evaluate candidate drugs, select therapeutic antibodies and protein-based therapeutics, with preferred biological response functions. Candidate agents comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetic agents, etc. Compounds of interest include chemotherapeutic agents, anti-inflammatory agents, hormones or hormone antagonists, ion channel modifiers, and neuroactive agents. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

The term “genetic agent” refers to polynucleotides and analogs thereof, which agents are tested in the screening assays of the invention by addition of the genetic agent to a cell. Genetic agents may be used as a factor, e.g. where the agent provides for expression of a factor. Genetic agents may also be screened, in a manner analogous to chemical agents. The introduction of the genetic agent results in an alteration of the total genetic composition of the cell. Genetic agents such as DNA can result in an experimentally introduced change in the genome of a cell, generally through the integration of the sequence into a chromosome. Genetic changes can also be transient, where the exogenous sequence is not integrated but is maintained as an episomal agents. Genetic agents, such as antisense oligonucleotides, can also affect the expression of proteins without changing the cell's genotype, by interfering with the transcription or translation of mRNA. The effect of a genetic agent is to increase or decrease expression of one or more gene products in the cell.

Agents are screened for biological activity by adding the agent to cells in the system; and may be added to cells in multiple systems. The cells are then lysed, and the lysate spotted on an array, which is probed with a specific binding agent comprising a detectable marker.

In one aspect, agents that affect kinase or phosphatase activity are screened. Dor example, candidate agents may be assayed in the presence or absence of known kinase inhibitors or activators, and the lysates probed for a change in total or specific phosphotyrosine, phosphoserine, etc.

Kits for the practice of the invention are provided. Such kits may include suitable slides for spotting lysates, and a selection of suitable binding agents. Alternatively, pre-spotted cell lysates, e.g. a time course of T cells following antigenic stimulation, staged cancer cells, etc. may be provided, with or without specific binding agents. Generally such kits will include directions for the practice of the methods, and may further comprise suitable labels, buffers, controls, and the like.

The following examples are put forth for illustrative purposes, and are not intended to limit the scope of what the inventors regard as their invention. Unless indicated otherwise, parts are parts by weight, molecular weight is weight average molecular weight, temperature is in degrees Celsius, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. Accordingly, it should be understood that the scope of the invention is not limited by this detailed description, but by the appended claims as properly construed under principles of patent law.

### Experimental

To fabricate the protein microarrays, we used a contact-printing robotic microarrayer to deliver nanoliter (˜6 nl) volumes of whole cell lysates onto nitrocellulose-coated slides. FAST slides (Schleicher and Schuell BioSciences, Dassel, Germany). Depending on the experimental setup, two different formats of FAST slides were used. In experiments where the number of samples was greater than the number of analytes measured, slides with a single pad capable of holding over 2000 lysate spots were used. In our screening experiments where the number of analytes exceeds the number of samples, we used slides carrying eight sectored pads or subarrays for multiplex analysis. Under our current configuration, a maximum of 24 spots can be fitted onto each individual pad (6 mm×6 mm).

For small-scale experiments, we used a precision 8-pin manual microarrayer system (Microcaster system from Schleicher & Schuell). The resulting features measured approximately 500 μm in diameter. The protein lysates dried within seconds of printing and were tightly bound to the nitrocellulose platform via electrostatic interactions. After blocking the slides, they were probed with either phospho-specific or phosphorylation-state independent primary antibodies. Bound primary antibodies were detected with a horseradish peroxidase (HRP)-linked secondary antibody. To amplify the signal, we took advantage of tyramide signal amplification technology (Bobrow et al. (1992) J Immunol Methods 150:145) in which HRP catalyzes the deposition of a biotinyl-tyramide conjugate onto the slide. Bound biotin was detected with Cy3-labeled streptavidin and fluorescent intensity measured using a conventional DNA microarray scanner. Since variations can occur during spotting and sample preparation, we normalized the signal intensity of each spot to the level of β-actin, which serves as an internal marker for total protein deposited.

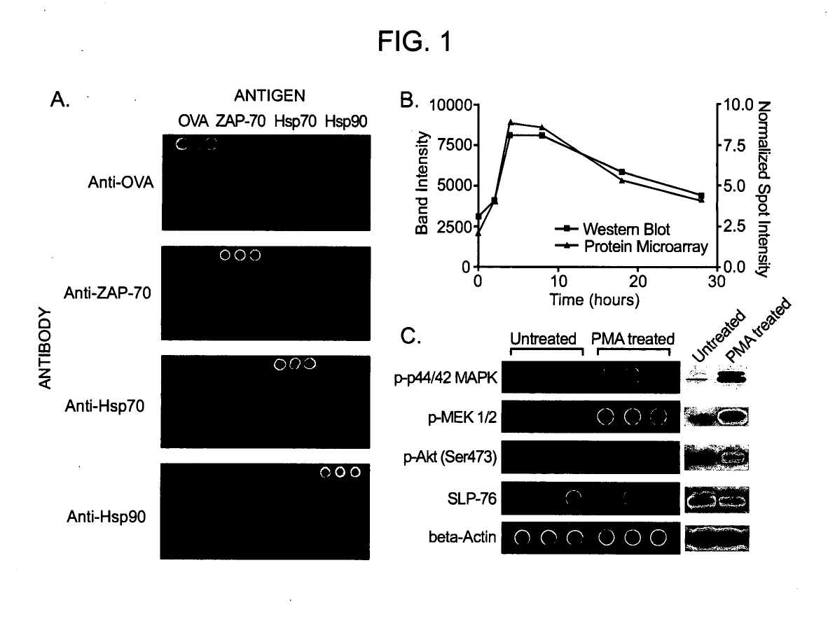

To test the sensitivity, dynamic range, and signal reproducibility of the arrays, we generated microarrays spotted with four purified antigens in triplicates over a range of 2-fold dilutions and probed the slides with their corresponding antibodies. FIG. 1A shows a representative image of the arrays. The limit of detection (defined as three standard deviations above background noise) per spot was determined to be 12 fg, 6 fg, 6 fg, 30 fg for ovalbumin, ZAP-70, Hsp70 and Hsp90, respectively. This level of sensitivity exceeds that of conventional immuoblotting methods. Sensitivity of HRP-catalyzed chemiluminescent detection methods is in the range of low picogram levels per band. Quantitative analysis revealed a linear dynamic range of at least 2 logs for all the antigens tested and a coefficient of variation of less than 10 percent for the majority (>90%) of replicates analyzed (data not shown). While the performance of the array appeared robust for purified antigens, it was important to validate its performance for analytes in a background of non-specific proteins such as whole cell lysates. We compared quantitative data generated from RRP microarrays with results obtained from immunoblotting measuring the kinetics of Hsp70 induction following heat shock in Jurkat T cells (clone E6-1). Both methods generated nearly identical results validating the use of RRP arrays for studying cell lysate samples (FIG. 1B).

To test if the array is sufficiently sensitive to detect changes in phosphorylation in an epitope specific manner, we generated arrays composed of lysates from either phorbol 12-myristate 13-acetate (PMA) treated or untreated Jurkat T cells and probed them with a panel of phospho-specific antibodies. PMA is known to directly activate intracellular protein kinase C (PKC) leading to the rapid phosphorylation of MEK1/2 and p44/42 MAPK. Phosphorylation of Akt/PKB, which is not linked to PKC-dependent pathways, is not significantly affected by PMA. As expected, we observed this pattern of phosphorylation using the microarrays (FIG. 1C).

Corresponding Western blots confirmed the results obtained by the arrays. Phosphorylation-state independent antibodies against SLP-76 and β-actin revealed no changes in the abundance of these two proteins, confirming the specificity of the observed changes in phosphorylation in MEK1/2 and p44/42 MAPK. The total amount of protein deposited on each spot was estimated to be equivalent to that of 20 to 30 cells, whereas 50,000 cell lysate equivalents were loaded per lane for Western blot analysis. Therefore RRP microarrays are especially suited to study rare cell populations where only small amounts of sample can be obtained for experimentation.

RRP microarrays have many potential applications in the study of signal transduction. Information on the kinetics of phosphorylation and dephosphorylation of a signaling protein often provides insight into how a signaling pathway is regulated and the activity of upstream kinases and phosphatases. Since a large number of samples collected at different time points and stimulation conditions must be analyzed to generate an informative database, protein microarrays are ideally suited for this task. As a first application, we used the arrays to profile the kinetics of phosphorylation of phospholipase C (PLC) γ1 on tyrosine 783 in T cells activated through their membrane CD3 and CD28 receptors. Phosphorylation by Syk at tyrosine 783 activates the enzymatic activity of PLCγ1 and thus serves as a useful indicator of PLCγ1 activity.

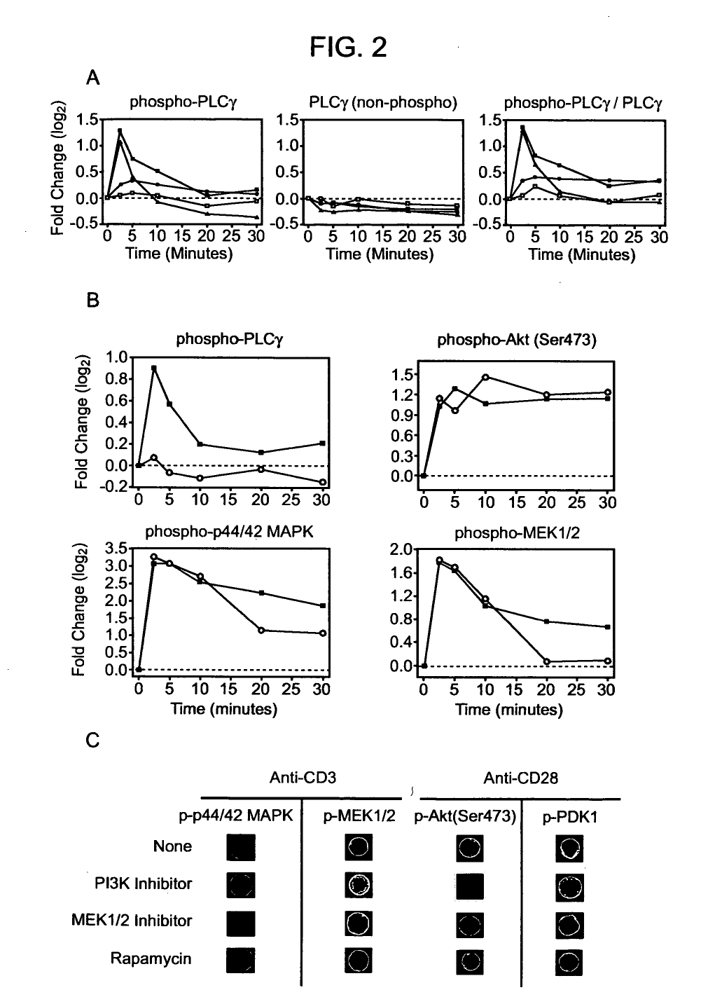

Lysates from Jurkat T cells stimulated with varying combinations of crosslinking anti-CD3 (UCHT1), anti-CD28 (9.3) and isotype control antibodies were collected over a 30-minute time course, spotted onto slides, and probed with a phospho-PLCγ1 (Y783) specific antibody (FIG. 2A, left panel). To obtain ratiometric data normalized to total PLCγ1 level, we probed an identical array with a phosphorylation-state independent PLCγ1 antibody (FIG. 2A, middle panel) and calculated the ratio of phospho-PLCγ1 to total PLCγ1 (FIG. 2A, right panel). As anticipated, CD3 crosslinking alone resulted in a rapid increase in the level of PLCγ1 phosphorylation within the first 2.5 minutes of stimulation. However, this level of phosphorylation was not sustained and quickly diminished to baseline by 10 minutes. CD28 stimulation alone contributed to a lesser but sustained increase in PLCγ1 phosphorylation lasting at least 30 minutes. In combination with CD3 crosslinking, CD28 costimulation prevented the level of phosphorylation from diminishing to baseline and maintained it at a level comparable to that of CD28 stimulation alone. This observation suggests that CD28 costimulation facilitates optimal T cell activation by sustaining PLCγ1 activity. This model is supported by several recent studies showing amplified PLCγ1 activation, Ca²⁺ flux, and NFAT-mediated transcriptional activity following CD28 costimulation.

The study of signaling pathways has been greatly aided by cell lines deficient in specific signaling molecules. To demonstrate the utility of protein microarrays in the delineation of signaling pathways, we stimulated J.gamma1 cells, a mutant line of Jurkat T cells deficient in PLC-γ1, and wildtype Jurkat T cells with both anti-CD3 and anti-CD28 antibodies and compared the kinetics of phosphorylation on four signaling proteins (FIG. 2B). As anticipated, we observed a rapid increase in the phosphorylation of PLCγ1 within minutes of stimulation followed by a gradual decline in wildtype cells. No change in signal was detected for J.gamma1, confirming the deficiency of the protein in the mutant line. The absence of PLCγ1 did not affect the kinetics of Akt phosphorylation at serine 473. In contrast, the rate of dephosphorylation for MEK1/2 and p44/42 MAPK was significantly faster in J.gamma1 cells but the initial peak in phosphorylation was not appreciably different. These results suggest that PLCγ1 may play a role in sustaining the activation of the p44/42 MAPK kinase pathway in T cells. In fact, previous studies have also demonstrated the dependence of p44/42 MAPK activation on Ca2+ flux and PLCγ activity, confirming our finding of crosstalk between the two pathways.

The recent success of imatinib mesylate (STI 571 or Gleevec) in the treatment of bcr/abl positive chronic myelogenous leukemia highlights the potential for specific kinase inhibitors in the treatment of a variety of diseases, including cancer and autoimmune diseases. High-throughput technologies capable of screening a large library of compounds for kinase inhibition activity will be crucial in the development of these drugs.

To demonstrate the application of protein microarrays in drug screening, we performed an experiment with three well-characterized pharmacological kinase inhibitors including LY294002 (phosphatidylinositol 3 (PI3) kinase inhibitor), U0126 (MEK1/2 inhibitor), and rapamycin (FRAP/mTOR inhibitor). Arrays composed of lysates from Jurkat cells stimulated through CD3 or CD28 in the presence of one of the inhibitors were probed with four different phospho-specific antibodies. As shown in FIG. 2C, U0126 specifically blocked the phosphorylation of p44/42 MAPK but had no effect on the phosphorylation of the other 3 kinases including MEK1/2, the upstream kinase of p44/42 MAPK. LY294002 reduced the phosphorylation of Akt (Ser473), a downstream target of PI-3-kinase but again had no effect on phosphorylation of the other 3 kinases. Since thousands of samples can be spotted onto one slide surface, a large compound library can theoretically be screened in a limited number of protein microarray slides. This demonstration illustrates the potential of this technology for screening lead compounds in the development of targeted therapeutics.

The identification of downstream signaling events associated with receptor stimulation is a challenging process using currently available techniques. The ability to monitor the phosphorylation state of numerous signaling proteins in parallel makes protein microarrays an attractive tool for this task. To demonstrate its application in identifying novel signaling events, we employed the microarrays to profile the phosphorylation state of 62 different signaling components in Jurkat T cells stimulated with anti-CD3 alone and in combination with anti-CD28.

Lysates collected 2.5 mins following stimulation were spotted in six replicates onto slides in an 8-pad subarray format and probed with a panel of phospho-specific antibodies. A change in phosphorylation was considered significant if it fulfilled four stringent inclusion criteria. The four criteria are: 1) average signal-to-noise ratio for the six replicate spots must be greater than 2 (screen #1) or 1.6 (screen #2); 2) Log2(fold change)>0.2; 3) Student t-test: p-value>0.05; 4) Significant in both screen #1 and #2.

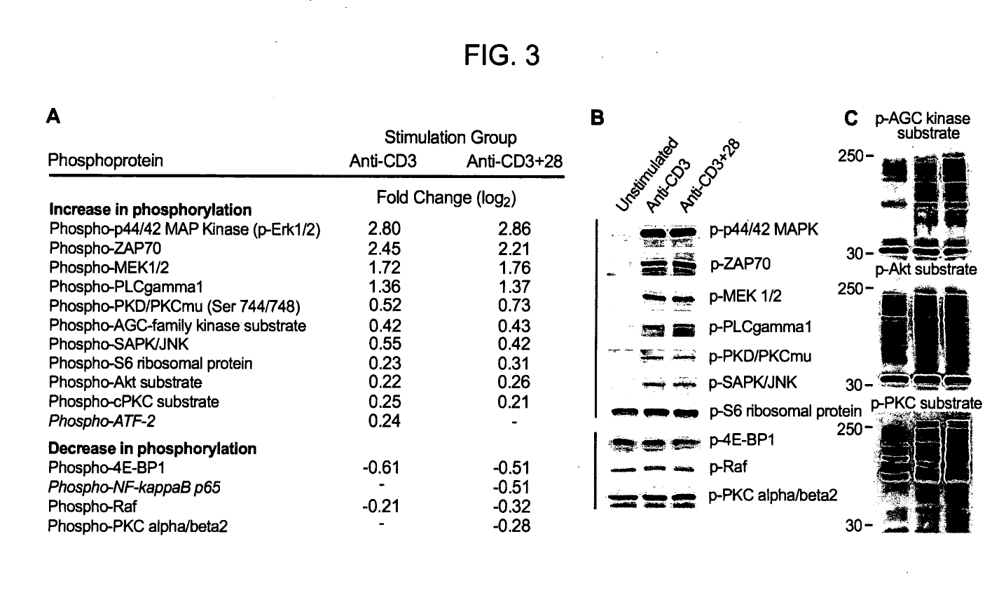

Of the 62 phosphoproteins probed, 13 signaling proteins experienced a significant change in phosphorylation with CD3 stimulation alone, and 14 proteins with CD3 and CD28 costimulation (FIG. 3A). The two lists overlapped significantly with each other, suggesting that CD28 costimulation does not significantly modify the signaling pathways that become activated shortly after TCR stimulation. The changes detected using the arrays were individually validated by performing conventional Western immunoblots (FIG. 3, B and C). Two of the phosphoproteins, p-ATF-2 and p-NF-kappaB p65, revealed no detectable bands of the correct molecular weight on their immunoblots.

The immunoblot analyses confirmed the changes in phosphorylation for the remaining detectable phosphoproteins. Many of the phosphoproteins on the list are well known to be involved in T cell receptor (TCR) signaling including p44/42 MAPK (Erk 1/2), ZAP-70, MEK1/2, PLCgamma1 and SAPK/JNK. Protein kinase D (PKD/PKC mu) has recently been shown to be activated following TCR stimulation and it appears to be a downstream target of PKC theta. Phosphorylation of S6 ribosomal protein by p70 S6 kinase is correlated with an increase in translational activity and TCR stimulation has been reported to activate the enzymatic activity of p70 S6 kinase. Several of the phospho-specific antibodies on the list recognized phosphorylated substrate motifs of kinases including cPKC, Akt and AGC-family kinases, indicating upregulated activity of those kinases following TCR stimulation.

Interestingly, several dephosphorylation events were detected. An unexpected finding was the dephosphorylation of Raf-1 at serine 259. This signaling event has not been previously reported to be associated with TCR stimulation. Phosphorylation at serine 259 negatively regulates Raf-1 activity since mutation of Ser259 to alanine generates a constitutively active kinase. Furthermore, Akt has been reported to inhibit Raf-1 activity by phosphorylating Ser259. However the precise mechanism by which Ser259 phosphorylation downregulates Raf-1 activity is unclear. Nevertheless it makes sense for the TCR to promote Raf-1 dephosphorylation at Ser259 since the ERK/MAPK pathway, which requires Raf-1 activity for optimal activation, is strongly stimulated upon TCR crosslinking.

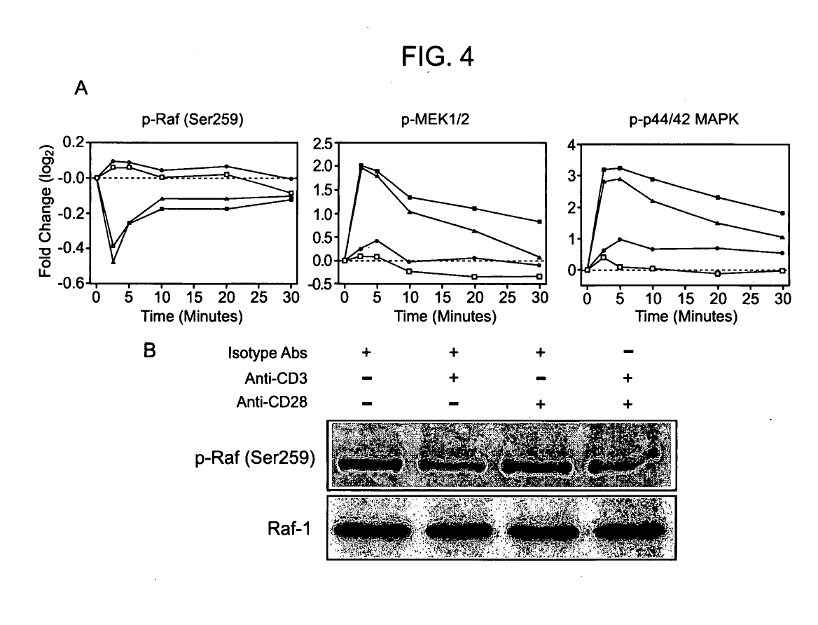

Since Raf-1 is the upstream kinase of MEK, we further investigated whether the dephosphorylation of Raf-1 coincided with its ability to phosphorylate MEK1/2. We used the arrays to profile the phosphorylation kinetics of Raf-1, MEK1/2 and p44/42 MAPK in parallel over a 30-minute stimulation period (FIG. 4A). We observed that Ser259 dephosphorylation occurred rapidly following CD3 stimulation and peaked at 2.5 minutes. Ser259 partially re-phosphorylated in the next 5 to 10 minutes and maintained at a sub-baseline level of phosphorylation for the remaining stimulation period. CD28 crosslinking alone had no appreciable effect on Ser259 dephosphorylation, and even in the setting of costimulation, Raf-1 dephosphorylation was insensitive to CD28. The kinetics of MEK1/2 and p44/42 MAPK phosphorylation followed closely with that of Raf-1 dephosphorylation, suggesting that Raf-1 activity is regulated through Ser259 in concordance with previously published data.

To confirm this signaling event is a genuine physiological response in normal T cells, we stimulated purified peripheral human T lymphocytes with anti-CD3 and anti-CD28 and determined the level of Ser259 phosphorylation through immunoblotting. A similar response was observed in primary T cells when stimulated for 20 minutes (FIG. 4B). Surprisingly, no dephosphorylation was observed with a 5-minute stimulation suggesting that the kinetics of dephosphorylation may be faster in transformed leukemic cells. Protein phosphatases 1 and 2A are likely to be involved in catalyzing the actual dephosphorylation reaction.

Traditional protein analysis methods have provided invaluable insight into signal transduction pathways; however, new proteomics approaches are necessary to unravel the complexity of the largely-unexplored proteome. RPP microarrays represent an important weapon in this armamentarium. In addition to the applications described above, RPP microarrays can be used to (i.) analyze the proteome of rare cell populations such as tumor cells, autoreactive lymphocytes purified using tetramers, or specialized cells isolated from other organisms such as Drosophila and C. elegans; (ii.) identify “cryptic” signaling pathways or defects revealed by exposing living tumor cells or other disease tissues to cytokines, chemokines or other biomolecules; (iii.) screen large panels of monoclonal antibodies for their ability to recognize stimulus-specific signaling molecules or inducible proteins; and (iv.) identify stimulus-specific changes in subcellular localization of biomolecules by spotting fractionated cell lysates. By combining genomic profiling technologies with RPP microarray platforms, it is now be possible to piece together the complex pathways connecting the cell surface, genome, and proteome, both in health and in disease.

The above data demonstrate the application of RPP microarrays to the study of signaling kinetics, pathway delineation, inducible protein identification, and high-throughput drug compound screening. RPP microarrays were employed to profile the phosphorylation state of 62 signaling components in Jurkat T lymphocytes stimulated through their membrane CD3 and CD28 receptors, identifying a novel link between CD3 crosslinking and dephosphorylation of Raf-1 at serine²⁵⁹. RPP microarrays, prepared using simple procedures and standard microarray equipment, represent a powerful new tool for proteomics and systems biology research.

In addition to the applications described above, RPP microarrays can be used to analyze the proteome of rare cell populations such as tumor cells, autoreactive lymphocytes purified using tetramers, or specialized cells isolated from other organisms such as Drosophila and C. elegans; identify “cryptic” signaling pathways or defects revealed by exposing living tumor cells or other disease tissues to cytokines, chemokines or other biomolecules; screen large panels of monoclonal antibodies for their ability to recognize stimulus-specific signaling molecules or inducible proteins; and identify stimulus-specific changes in subcellular localization of biomolecules by spotting fractionated cell lysates. By combining genomic profiling technologies with RPP microarray platforms, it is possible to piece together the complex pathways connecting the cell surface, genome, and proteome, both in health and in disease.

1. A method of determining a pattern of response to an agent, the method comprising:
contacting a cell with said agent;
preparing a lysate of said cell before and after said contacting step;
spotting said lysate on a microarray;
probing said microarray with a reagent that specifically recognizes a post-translational modification of a polypeptide of interest;
determining the alteration in post-translational modification as a result of said contacting with said agent.
